(19) United States
(12) Patent Application Publication
(10) Pub. No.: US 2025/0127394 A1
(43) Pub. Date: Apr. 24, 2025

(54) Line-field OCT System with Radial Scanning

(71) Applicant: KineoLabs, Inc., Billerica, MA (US)

(72) Inventors: Yisi Liu, Winchester, MA (US); Walid A. Atia, Jamaica Plain, MA (US); Dale C. Flanders, Lexington, MA (US)

(21) Appl. No.: 18/918,754

(22) Filed: Oct. 17, 2024

Related U.S. Application Data
(60) Provisional application No. 63/592,004, filed on Oct. 20, 2023.

(51) Int. Cl. A61B 3/10

(52) U.S. Cl. CPC A61B 3/102

(57) ABSTRACT

A line-field parallel swept optical coherence tomography (OCT) system optimized for high-resolution ophthalmic imaging and capable of broad industrial applications. The system employs a gain chip using gallium-aluminum-arsenide (GaAlAs) for light amplification within a specific “water window” wavelength range, suitable for deep tissue imaging. The design incorporates a hermetically sealed packaging with an optional thermoelectric cooler and utilizes a single angled facet (SAF) with high reflectivity and antireflective coatings to enhance laser performance. The optical path includes a collimating lens, a cat's eye focusing lens, and a bandpass filter adjustable via an angle control actuator for dynamic wavelength tuning. The system features a rotator-derotator mechanism utilizing Dove prisms or k-mirror devices, for example, for precise radial scanning. This allows for quick, accurate imaging, making it ideal for capturing high-resolution images of the retina and other surfaces.

### RELATED APPLICATIONS

This application claims the benefit under 35 USC 119 (e) of U.S. Provisional Application No. 63/592,004, filed on Oct. 20, 2023, which is incorporated herein by reference in its entirety.

### BACKGROUND OF THE INVENTION

Optical coherence tomography (OCT) is a cross-sectional, non-invasive imaging modality that is used in many areas of medical imaging. For example, in ophthalmology, OCT has been widely used for imaging the retina, choroid and anterior segment. Functional imaging of the blood velocity and vessel microvasculature is also possible.

Fourier-domain OCT (FD-OCT) has recently attracted more attention because of its high sensitivity and imaging speed compared to time-domain OCT (TD-OCT), which uses an optical delay line for mechanical depth scanning with a relatively slow imaging speed. The spectral information discrimination in FD-OCT is accomplished either by using a dispersive spectrometer in the detection arm (spectral domain or SD-OCT) or rapidly scanning a swept laser source (swept-source OCT or SS-OCT).

Compared to spectrometer-based FD-OCT, swept-source OCT (SS-OCT) has several advantages, including its robustness to motion artifacts and fringe washout, lower sensitivity roll-off and higher detection efficiency.

Many different approaches have been implemented to develop high-speed swept sources for SS-OCT. One approach employs a semiconductor optical amplifier (SOA) based ring laser design (see for example Yun et al. “High-speed optical frequency-domain imaging” Opt. Express 11:2953. Short cavity lasers (see for example Kuznetsov et al. “Compact Ultrafast Reflective Fabry-Perot Tunable Lasers For OCT Imaging Applications,” Proc. SPIE 7554: 75541F 2010) are another example. SOA based ring laser designs have been practically limited to positive wavelength sweeps (increasing wavelength) because of the significant power loss that occurs in negative tuning. This has been attributed to four-wave mixing (FWM) in SOAs causing a negative frequency shift in intracavity light as it propagates through the SOA (Bilenca et al “Numerical study of wavelength-swept semiconductor ring lasers: the role of refractive-index nonlinearities in semiconductor optical amplifiers and implications for biomedical imaging applications,” Opt. Lett. 31:760-762 2006).

At the same time, other architectures exist for SS-OCT that reduce the performance requirements for the swept laser source. Fechtig et al. in an article entitled Line-Field parallel swept source MHz OCT for structural and functional retinal imaging, Biomedical Optics Express 716, vol. 6, no. 3, (2015) describes a system that achieves 1 MHz equivalent A-scan rates by combining a lower sweep rate laser with a linear sensor. Even earlier examples exist such as Line-Field Optical Coherence Tomography Using Frequency-Sweeping Source by Lee et al. in IEEE Journal of Selected Topics in Quantum Electronics, Vol. 14, No. 1, January 2008.

### SUMMARY OF THE INVENTION

Commercial ophthalmic swept source OCT systems are traditionally flying spot systems. The systems scan the eye, resolving an A-scan at every point. A volume is built up typically by raster scanning the spot across the eye, typically the retina. The raster scanning is accomplished with an orthogonal pair of galvanometers.

Raster scanning is generally not a preferred option for a line-field system, but a single scanning galvanometer can be used to scan the line in a direction that is orthogonal to the extent of the line to thereby build up the desired volume.

The present invention concerns a different approach for scanning the line in a line-field system. An image rotator is employed to rotate the line on the sample, such as the retina of an eye. The image rotator unrotates the light returning from the sample so that sample light is properly interfered with the light from the reference arm on the line-field sensor.

The present invention relates to an optical coherence tomography (OCT) system designed to perform high-resolution imaging through radial scanning of a light beam across a sample using a rotator-derotator mechanism. The system comprises a light source configured to emit a beam of light and an optical assembly that manages the propagation of the beam both before and after interaction with the rotator-derotator mechanism. This configuration allows for precise control over the scanning process, enhancing image quality and resolution.

In one embodiment, the rotator-derotator mechanism includes one or more optical elements selected from the group consisting of Dove prisms and k-mirror devices. These optical elements are rotated by a control unit to alter the angle of the emitted beam, facilitating comprehensive scanning of the sample. The mechanism is designed to maintain the orientation of incoming and outgoing light beams, which can be configured through on-axis alignment for a 180-degree scanning range or off-axis alignment for a 360-degree scanning range. This flexibility enables the system to adapt to various imaging requirements and sample geometries.

An encoder is associated with the rotator-derotator mechanism to provide real-time feedback on the rotational position of the optical elements. A processor synchronizes the rotational adjustments of the rotator-derotator mechanism with image capture, ensuring that each positional change corresponds accurately with data acquisition phases. This synchronization optimizes imaging resolution and field of view, as the system can adjust scanning parameters dynamically based on the encoder's feedback.

The rotator-derotator mechanism may also include a feature to adjust the light beam's profile by modulating its cross-sectional shape and orientation during rotation. This adjustment enhances the resolution and field of view of the system, allowing for more detailed tomographic imaging. The mechanism interacts directly with line-forming optics and a beamsplitter to optimize the spatial distribution of the light. By precisely controlling the beam profile, the system improves imaging properties such as resolution and depth focus.

In addition to the hardware configurations, the invention encompasses methods for radial scanning in an OCT system. One method involves emitting a beam of light from a laser source and rotating it using the rotator-derotator mechanism to scan radially across a sample. Interference patterns resulting from the interaction of the scanned beam with the sample are detected to generate imaging data. Rotating the beam includes controlling the rotation of a Dove prism or a k-mirror device within the rotator-derotator mechanism and synchronizing the angle of rotation with data capture phases. This synchronization ensures optimal imaging resolution and field of view.

The method may further include adjusting the cross-sectional profile of the light beam before and after rotation to modulate imaging properties. Feedback from an encoder linked to the rotator-derotator mechanism is employed to adjust the beam orientation precisely. The system can dynamically alter the orientation of the beam in response to detected changes in sample characteristics or desired imaging areas. A control algorithm calculates optimal beam orientations based on real-time imaging feedback, allowing for adaptive scanning strategies.

Processing interference patterns in synchronization with rotational adjustments produces high-resolution cross-sectional images. A proportional-integral-derivative (PID) controller may be utilized to maintain desired beam characteristics and stability during scanning. Furthermore, the rotator-derotator mechanism can perform both rotational and translational movements to cover a comprehensive field of view of the sample. This capability is particularly beneficial for specific applications such as ophthalmic imaging or industrial material analysis, where detailed and extensive scanning is required.

In another embodiment, the OCT system includes a laser source configured to emit a beam of light with a tunable wavelength. A synchronization module coordinates the rotation of the rotator-derotator mechanism with the wavelength tuning of the laser source. This coordination optimizes imaging speed and resolution, as the system adjusts both spatial and spectral parameters concurrently. A detector array captures interference patterns resulting from the interaction of the rotated and wavelength-tuned beam with the sample.

The system may also feature a rotator-derotator mechanism capable of adjusting rotation speed and angle based on real-time feedback. A feedback sensor detects motion or characteristics of the sample, and a control unit adjusts the operation of the rotator-derotator mechanism accordingly. This adaptive adjustment enhances image stability and quality by compensating for sample movement or variations in sample properties during imaging.

Another method involves performing OCT imaging by emitting a beam of light from a laser source and continuously or discontinuously rotating it using a rotator-derotator mechanism to scan across a sample. Interference data of the light reflected from the sample during rotation are captured and processed to reconstruct a real-time three-dimensional image of the sample. This method enables comprehensive imaging by covering various angles and depths, providing detailed structural information about the sample.

Overall, the invention provides an advanced OCT system and methods that enhance imaging capabilities through the use of a rotator-derotator mechanism. By integrating precise rotational control, synchronization with imaging processes, and adaptive adjustments based on feedback, the system achieves high-resolution and high-quality imaging suitable for a wide range of applications.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is schematic side views of a cat's-eye tunable laser in a line-field parallel swept OCT system;

FIG. 2 is a plot of the laser frequency, angle of the angle control actuator 132, power wet point reference signal 250, RIN or error signal for the collection of the line interference signals for a B-scan;

FIG. 3A is a schematic view of a Dove prism style image rotator-derotator; and

FIG. 3B is a schematic view of a k-mirror style image rotator-derotator;

FIGS. 4A and 4B are schematic diagrams showing the relationship between the light to be projected on the eye and the center of rotation of the rotator-derotator 280.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

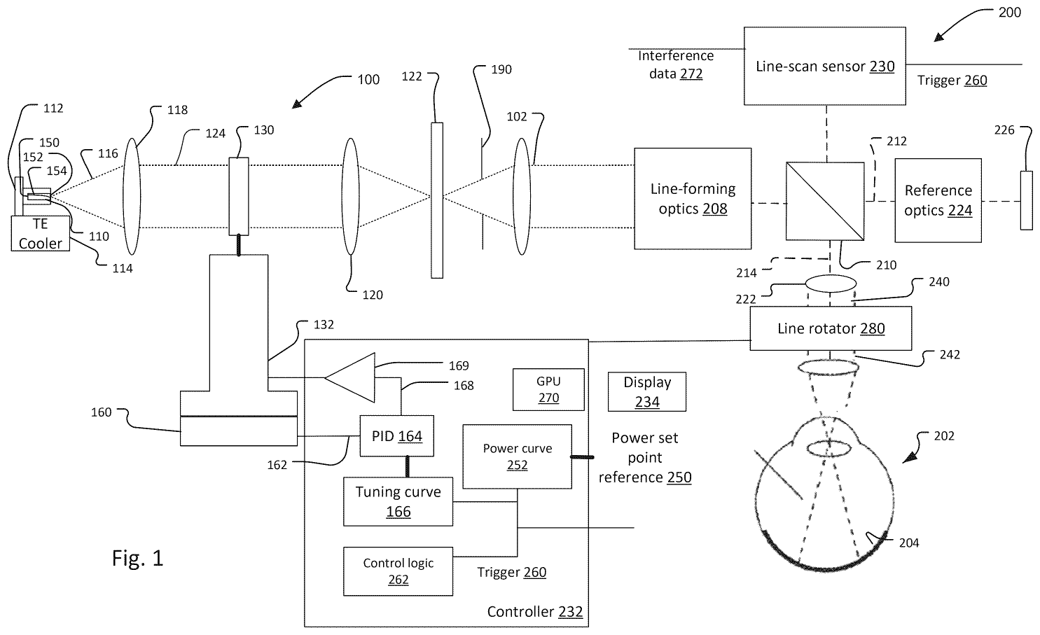

FIG. 1 shows a line-field parallel swept OCT system 200 with a line rotator, which has been constructed according to the principles of the present invention.

In the illustrated example, the laser's amplification is provided by a GaAlAs gain chip 110, in one example. The gain chip 110 amplifies light in the wavelength range of about 800 to 900 nanometers. Preferably its center wavelength is around 840 nanometers, which is useful for applications such as ophthalmic imaging and other diagnostic uses because of the water window (650 to 950 nm) at these wavelengths. Another advantage of this wavelength range is that it can be detected with standard cameras with silicon-based imager chips. Specifically, the output is detected with silicon, e.g., complementary metal-oxide-semiconductor (CMOS) or charge-coupled device (CCD), imagers.

Other material systems can be selected for the gain chip, however. Common material systems are based on III-V semiconductor materials, including binary materials, such as GaN, GaAs, InP, GaSb, InAs, as well as ternary, quaternary, and pentenary alloys, such as InGaN, InAlGaN, InGaP, AlGaAs, InGaAs, GaInNAs, GaInNAsSb, AlInGaAs, InGaAsP, AlGaAsSb, AlGaInAsSb, AlAsSb, InGaSb, InAsSb, and InGaAsSb. Collectively, these material systems support operating wavelengths from about 400 nanometers (nm) to 2500 nm, including longer wavelength ranges extending into multiple micrometer wavelengths. Semiconductor quantum well, quantum cascade and quantum dot gain regions are typically used to obtain especially wide gain and spectral emission bandwidths, and support operation up to 250 μm in wavelength. Quantum well layers may be purposely strained or unstrained depending on the exact materials and the desired wavelength coverage.

In the preferred current embodiment, the gain chip 110 is mounted in a TO-can type hermetic package 112. This protects the chip 110 from dust and the ambient environment including moisture. In some examples, the TO-can package has an integrated or a separate thermoelectric cooler 114.

The chip 110 is preferably a single angled facet (SAF) edge-emitting chip. As such, it has a high reflectivity (HR) coated rear facet 150. It has an antireflective (AR) coated front facet 152. In addition, for improved performance, it has a curved ridge waveguide 154 that is perpendicular to the rear facet 150 but is angled at the interface with the front facet 152. This angling at the front facet along with the AR coating reduces reflections at the front facet reflectivity by up to 40 dB and significantly improves laser performance by reducing parasitic reflections that can otherwise lead to non-smooth tuning and mode-hopping.

The free space beam 116 from the package 112 is diverging in both axes (x, y). It is collimated by a collimating lens 118. The resulting collimated beam is received by a cat's eye focusing lens 120, which focuses the light onto a cat's eye mirror/output coupler 122. This defines the other end of the laser cavity, extending between the mirror/output coupler 122 and the back/reflective facet of the gain chip 110.

The collimated light 124 between the collimating lens 118 and the cat's eye focusing lens 120 passes through a thin film interference bandpass filter 130. This provides a pass band of approximately 0.3 nanometers (nm) full width at half maximum (FWHM) for OCT applications. More generally, its pass band is between 0.2 nm and 0.5 nm FWHM, or more generally between 0.1 nm and 2 nm FWHM. Even more generally, it is between 0.05 nm to 5 nm FWHM.

The bandpass filter is held on an arm of an angle control actuator 132 that changes the angle of the bandpass filter 130 to the collimated light 124. In one example, the angle control actuator is a galvanometer. In other examples, the angle control actuator 132 is a servomotor or an electrical motor that continuously spins the bandpass filter 130 in the collimated beam 124. This allows for tilting of the bandpass filter 130 with respect to the collimated beam 124 to thereby tilt-tune the filter and thus change the passband to scan or sweep the wavelength of the swept laser 100.

Tuning speed specifications for a galvanometer generally range from 0.1 Hz to 50 KHz. For the higher speeds, a 25 kHz resonant galvanometer can be used with bi-directional tuning, but higher and lower speeds can be used. Wavelength tuning speed is usually given in nm/sec, so for a 100 Hz tuning speed ideal for retinal imaging applications where a line-speed camera at 100 kHz will give 1000 sampled bandwidth points and 70 nm tuning range, this would give 70 nm/10 msec=7000 nm/sec. In general, the tuning speed should be between 3,000 nm/sec and 11,000 nm/sec or higher.

For retinal or industrial imaging with low-cost CMOS or CCD cameras, 840 nm center wavelength is an ideal water window. The tuning range is usually minimally 30 nm of tuning range. Preferably, the tuning range is closer to 60 nm or 70 nm or more. This provides good resolution of <8 micrometers in air. In general, the tuning range should be between 30 nm and 100 nm.

The size of the collimated beam 124 is important for many applications. As a general rule, a smaller beam results in higher divergence resulting in a larger cone half angle (CHA). This reduces the minimum line width over angle for a tunable filter. In the current embodiment, the collimated beam is preferably not less than, i.e., greater than, 1 millimeter (mm) full width half maximum (FWHM) and is preferably greater than 2 mm FWHM for retinal OCT application. It can be smaller, however, for many spectroscopy applications in the infrared, visible or ultraviolet. In general, the CHA should be less than 0.04×0.02 degrees and preferably about 0.02×0.01 degrees or less.

The light from the gain chip is polarized. In the common architectures, the polarization is horizontal or parallel to the epitaxial layers of the edge-emitting gain chip 110. In the preferred configuration, the filter is oriented to receive the S polarization in order to maintain narrow line width of the filter as it is tilt tuned. On the other hand, the P polarization broadens drastically at large tilt angles. S polarization has higher loss at larger tilt angles than P. So, the filter design needs to address these issues by providing a low enough loss across the tuning band for S, in the current embodiment.

In general, the present cat's-eye configuration provides a number of advantages. It provides low loss, low tolerance, repeatable stable operation since it provides for a lower angle wavelength change over grating-based lasers.

The mirror/output coupler 122 will typically reflect about 80% of the light back into the laser's cavity and transmit about 20% of light. More generally, the mirror/output coupler can reflect from 10% to 99% of light (transmitting 90% to 1%, respectively), depending on the output power and laser cavity loss desired. Higher reflectivity results in lower loss cavities and thus a wider laser tuning range where gain exceeds loss, but results in lower output power. In typical operation, the mirror/output coupler 122 reflects less than 90%.

In some embodiments, an iris or mask 190 is added typically after the mirror output coupler 122 to clip the beam edge. This reduces power fluctuations as the beam wanders due to refraction in the tilting bandpass filter 130. Preferably, it is between 80% and 95% and preferably about 90% of the beam size. In other examples, the iris or mask 190 is added in the laser cavity or after the laser cavity to a collimated portion of the beam.

Typically, the diverging beam from the mirror output coupler 122 is typically collimated with an output collimating lens 140 to form a free space output beam 102.

The angle control actuator 132 is operated as a servomechanism. In the illustrated embodiment, the angle control actuator 132 is a servo controlled galvanometer with an encoder 160. The encoder 160 produces an angle signal 162 indicating the angle of the galvanometer and thus the filter 130 to the collimated beam 124. Preferably, the encoder is an optical encoder and is often analog.

A controller 232 receives the angle signal 162 at a PID (proportional-integral-derivative) controller 164. The PID controller 164 compares the angle signal 164 to a specified tuning function stored in the tuning curve module 166. Often, the desired tuning curve is stored in a look up table or is generated algorithmically. Often this is an approximately sawtooth or triangular waveform. The PID controller 166 produces the control function 168 that is used to drive the windings of the galvanometer 132 via an amplifier 169.

In the illustrated example, the OCT system 200 is employed for ophthalmic analysis of a human eye 202 and specifically the retina 204, in a current implementation. That said, the system can also be used for analysis of other samples, both living and non-living.

Light in the form of free space beam 102 from the laser 100 passes in free space to line-forming optics 208 and then to a beamsplitter 210, such as a cube beamsplitter, of the OCT interferometer.

Typically, the line-forming optics 208 includes one or more cylindrical lenses and possibly several additional lenses in a beam expander configuration. The line forming optics 208 converts the light from the laser 100 into a line or more specifically a rectangular profile with an aspect ratio of at least 10 to 1 and typically greater than 100:1, and often 400:1, or more measured at the FWHM. That is, when looking along its optical axis, the light from the line-forming optics 208 has a line or more specifically a rectangular two-dimensional profile that is at least 10 times longer in one dimension than the other dimension, measured at the FWHM.

The beamsplitter 210 divides the light between the reference arm 212 and the sample arm 214 in the illustrated Michelson arrangement. The light propagates in free space between one or more lenses that form projection and collection optics 222 in the sample arm and illuminates the sample 202, a typical sample being tissues, e.g. retina, 204 in the human eye.

According to the invention, the line of light is radially scanned across the sample with a line rotator-derotator 280.

Under the control of the controller 232, The rotator-derotator 280 rotates its input line-shaped light 240 so that the line it outputs 242, and as projected onto the sample 202, is rotated. At the same time, the scattered light from the sample that is coupled into the rotator-derotator 280 is derotated so that the orientation matches the light coming into the rotator-derotator 280, according to Helmholtz reciprocity.

On the other hand, light in the reference arm 212 is conditioned by one or more lenses of reference arm optics 224 and reflected by reference mirror 226.

The collected sample light received back through the projection and collection optics 222 is combined with reference arm light to form light interference in a line-field camera or sensor 230. The line field sensor typically has a linear array of at least 512 pixels, and often at least 1024 or 2048 pixels to detect interference signals for a line. Often the line is only 1 pixel wide in the lateral direction. In a current example, however, the linear array is a few pixels wide such as between 2 and 10 pixels wide. Often the pixels can be binned in this lateral direction for higher sensitivity.

In some embodiments, the controller analyzes the information collected by the line-field camera or sensor 230 to function as a feedback sensor configured to detect motion or characteristics of the sample and/or quality of the collected interference data and/or determined images and adjusts the scanning of the sample accordingly.

An important aspect of the illustrated example is that the light from the cats-eye swept laser 100 through the OCT interferometer to the line-scan sensor 230 travels in free space between the cube beamsplitter 210, and the lenses of the line-forming optics 208, collection optics 222, reference arm optics 224 in freespace. No waveguides, such as optical fiber, need to be present.

The interference data 272 output from the sensor 230 is readout by a controller 232 and typically processed by a graphic processing unit GPU 270 or other processing unit into three-dimensional images. The results can be stored in the controller 232 and/or displayed on display 234. The Fourier transform of the interference light at the different wavelengths or frequencies of the swept laser 100 reveals the profile of scattering intensities at different path lengths, and therefore scattering as a function of depth (z-direction) in the sample (see for example Leitgeb et al, “Ultrahigh resolution Fourier domain optical coherence tomography,” Optics Express 12 (10): 2156 2004). The profile of scattering as a function of depth for a point is called an axial scan (A-scan). The combination of the projected line and line-scan sensor 230 produces a cross-sectional image (tomogram or B-scan) of the sample. As the projected line is rotated by the line rotator-derotator 280 radially around the retina or other sample by line rotator-derotator 280, a collection of B-scans are acquired creating a data volume or C-scan.

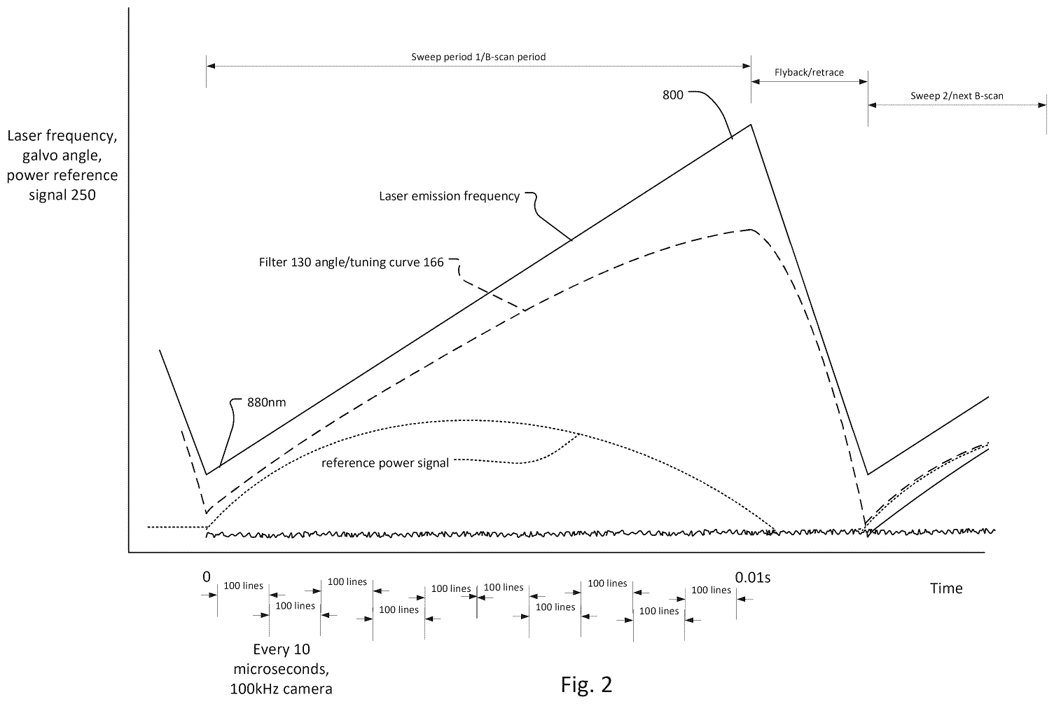

FIG. 2 is a plot of the laser frequency, angle of the angle control actuator 132, power reference set point signal 250, RIN or error signal for the collection of the line interference signals needed for a B-scan.

In more detail, the control logic 262 of the controller 232 triggers the beginning of the scan with the trigger 260. The laser 100 is swept in wavelength so that its frequency changes preferably linearly with time over the sweep or B-scan period. A linear frequency sweep is often desirable because it allows for efficient use of the camera sample rate and avoids the need to resample.

For the linear sweep in frequency, the angle of the filter 130 must be tuned in a non-linear fashion. As shown, the rate of change of the angle of the filter 130 slows with increasing angle and shorter wavelengths, higher frequency of the laser emission 102. This tuning curve for linear frequency sweeping is produced by and/or stored in the tuning curve module 166.

As the laser 100 begins its sweep, the control logic 262 of the controller 232 generates the trigger signal that initiates the capturing of the line interference signals as the laser tunes. Preferably, at least 250 line interference signals are captured by the line-scan camera 230 within the sweep period of the laser 100. Currently more than 500 line interference signals, such as 1000 line interference signals or more are captured.

Preferably, the sweep period is less than 0.05 seconds and preferably less than 0.02 seconds. In the current examples, a sweep period of about 0.01 seconds or less provides acceptable B-scans of the human eye despite microsaccades and other movement.

In a current implementation, the line-field camera 230 operates as about 100 kLPS (lines per second) or faster for a sweep period of about 0.01 seconds. This informs the requirements for the RIN cancellation. The RIN cancellation only needs to be within the Nyquist frequency of the line-scan camera line speed. For example, a 100 kLPS camera speed will have a Nyquist frequency of 50 kHz where the sampling is relevant. Therefore, the loop bandwidth of the feedback control of the ridge injection current is at least 10% of the line rate of the line-scan camera and is preferably about 50% of that line rate and can be 100% or more of the line rate.

Once several lines, typically more than 5 but often less than 50 lines, of interference data 270 have been captured and B-scans produced, the controller 232 controls the line rotator-derotator 280 to rotate by a predetermined angle such as a few degrees or up to 90 degrees. The process repeats and several lines are captured and B-scans produced for the new angle. At each angle, the several B-scans are often averaged to produce an averaged image for each angle. In one example, the line rotator-derotator 280 is rotated at increments of between 3 degrees and 15 degrees for a total of 180 degrees or +/−90 degrees to create a full 360 degree scan of the sample. In other examples, it rotates a full 360 degrees.

In another mode of operation, the rotator-derotator 280 is controlled to turn at a constant rate while the captured B-lines are synchronized to an encoder to allow for real-time 3D video. Typically the rate of rotation is less than 5 Hertz (Hz) and typically less than 1 Hz. In fact, it can be less than 0.5 Hz to prevent motion blur.

More generally, the controller dynamically alters the orientation of the beam by controlling the line rotator-derotator 280 based on its encoder in response to detected changes in sample characteristics or desired imaging areas. The controller employs a control algorithm to calculate optimal beam orientations based on real-time imaging feedback determined based on the detected interference data.

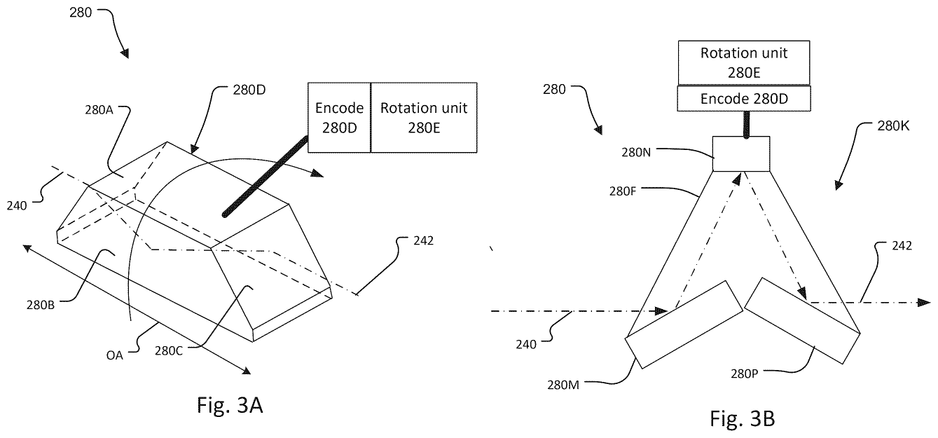

FIG. 3A shows one implementation of the rotator-derotator using a Dove prism 280D.

Typically, the Dove prism 280D is made from high-quality optical glass such as BK7 or fused silica. Its shape is rectangular with a trapezoidal cross-section, elongated along its length.

The dove prism 280D is oriented so that input light 240 and output light 242 are parallel to its long axis and the optical axis OA. The input light is refracted by the front face 280A. The light is then reflected, typically by total internal reflection, by the base face 280B and then is again refracted by the rear face 280C. The scattered light from the sample follows the reverse path and by the principle of reciprocity will match the angle of the input light 240.

A rotator unit 280E rotates the Dove prism 280D around an axis parallel to the input light 240, output light 242, and optical axis OA under control of the control logic 262.

In a preferred embodiment, the rotator unit 280E further includes an angle encoder 280D to enable the control logic to monitor its angle and register collected B-scan in angle to build volumes from the collected B-scans. Preferably the controller 232 implements PID control of the rotator unit 280E via the angle information from the angle encoder 280D.

FIG. 3B shows another implementation of the rotator-derotator 280 using a k-mirror device.

The k-mirror device 280K is oriented so that input light 240 and output light 242 is parallel to its long axis defined by beams 240 and 242. The input light is reflected by a first mirror 280M. The light is then reflected by a second mirror 280N and then is again reflected by a third mirror 280P. The three mirrors are supported by a frame 280F which is away from the optical path or axis. The scattered light from the sample follows the reverse path and by the principle of reciprocity will match the angle of the input light 240.

A rotator unit 280E engages with the frame 280F and rotates the k-mirror device 280K around an axis parallel to the input light 240 and output light 242 under control of the control logic 262.

In a preferred embodiment, either type of rotator unit 280E further includes an angle encoder 280D to enable the control logic to monitor its angle and register collected B-scan in angle to build volumes from the collected B-scans. Preferably the controller 232 implements PID control of the rotator unit 280E via the angle information from the angle encoder 280D.

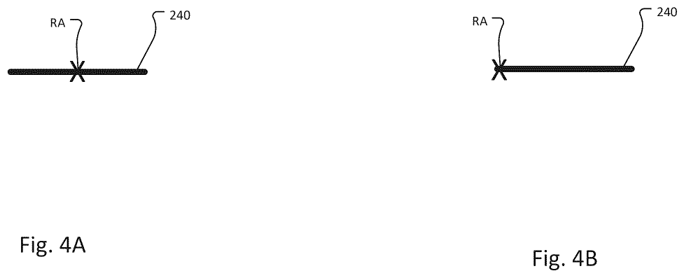

The rotator-derotator 280 can be employed in an on-axis arrangement. In this configuration the center of the line of light 240 received through lens 222 is parallel and coincident with the axis of rotation RA of the rotator-derotator 280 as shown in FIG. 4A. Specifically, the axis of rotation RA is located at a point between 40% and 60% of the length of the line 220. This configuration is useful because the rotator-derotator 280 only needs to rotate 180 degrees to scan the eye. The total width of the effective field of view is the length of the projected line of light 240 on the retina.

The rotator-derotator 280 can alternatively be employed in an off-axis arrangement as shown in FIG. 4B. In this configuration the one end of the line received through lens 222 is parallel and coincident with the axis of rotation of the rotator-derotator 280. Specifically, the axis of rotation RA is located at a point between 0% and 20% of the length of the line 220. In this configuration the rotator-derotator 280 needs to rotate a full 360 degrees to scan the eye. The total width of the field of view is up to twice the length of the projected line 240 on the retina, however.

While this invention has been particularly shown and described with references to preferred embodiments thereof, it will be understood by those skilled in the art that various changes in form and details may be made therein without departing from the scope of the invention encompassed by the appended claims.

What is claimed is:

1. An optical coherence tomography system, comprising:
a light source configured to emit a beam of light;
a rotator-derotator mechanism for radially scanning the beam of light across a sample;
an optical assembly configured to manage the propagation of the beam both before and after interaction with the rotator-derotator mechanism.

2. The system of claim 1, wherein the rotator-derotator mechanism comprises:
one or more optical elements selected from the group consisting of Dove prisms and k-mirror devices;
a control unit configured to rotate said optical elements to alter the angle of the emitted beam for scanning of the sample.

3. The system of claim 1, further comprising:
an encoder associated with the rotator-derotator mechanism, configured to provide feedback on the rotational position of the optical elements;
a processor programmed to synchronize the rotational adjustments of the rotator-derotator mechanism with image capture.

4. The system of claim 1, wherein the rotator-derotator mechanism is configured to maintain the orientation of incoming and outgoing light beams through on-axis alignment, facilitating a 180-degree scanning range.

5. The system of claim 1, wherein the rotator-derotator mechanism is configured to maintain the orientation of incoming and outgoing light beams through off-axis alignment, facilitating 360-degree scanning range.

6. The system of claim 1, wherein the rotator-derotator mechanism includes a mechanism to adjust the light beam's profile, enhancing the resolution and field of view of the system by modulating the beam's cross-sectional shape and orientation during rotation.

7. The system of claim 1, configured such that the rotator-derotator mechanism interacts directly with line-forming optics and a beamsplitter to optimize the spatial distribution of the light for detailed tomographic imaging.

8. A method for radial scanning in an optical coherence tomography system, the method comprising:
emitting a beam of light from a laser source;
rotating the beam of light using a rotator-derotator mechanism to scan radially across a sample; and
detecting interference patterns from the scanned beam to generate imaging data of the sample.

9. The method of claim 8, wherein rotating the beam of light includes:
controlling the rotation of a Dove prism or a k-mirror device within the rotator-derotator mechanism;
synchronizing the angle of rotation with data capture phases to optimize imaging resolution and field of view.

10. The method of claim 8, further comprising:
adjusting the cross-sectional profile of the light beam before and after rotation to modulate imaging properties such as resolution and depth focus;
employing feedback from an encoder linked to the rotator-derotator mechanism to adjust beam orientation precisely.

11. The method of claim 8, including:
dynamically altering the orientation of the beam in response to detected changes in sample characteristics or desired imaging areas;
employing a control algorithm to calculate optimal beam orientations based on real-time imaging feedback.

12. The method of claim 8, further comprising:
processing interference patterns in synchronization with rotational adjustments to produce high-resolution cross-sectional images;
utilizing a PID controller to maintain desired beam characteristics and stability during scanning.

13. The method of claim 8, further including:
configuring the rotator-derotator mechanism to perform both rotational and translational movements to cover a comprehensive field of view of the sample;
optimizing the scanning pattern for specific applications such as ophthalmic imaging or industrial material analysis.

14. An optical coherence tomography system comprising:
a laser source configured to emit a beam of light with a tunable wavelength;
a rotator-derotator mechanism configured to rotate the beam of light to scan across a sample;
a synchronization module that coordinates the rotation of the rotator-derotator mechanism with the wavelength tuning of the laser source to optimize imaging speed and resolution;
a detector array configured to capture interference patterns resulting from the interaction of the rotated beam with the sample.

15. An optical coherence tomography system comprising:
a laser source emitting a beam of light;
a rotator-derotator mechanism capable of adjusting rotation speed and angle based on real-time feedback;
a feedback sensor configured to detect motion or characteristics of the sample;
a control unit that adjusts the operation of the rotator-derotator mechanism in response to input from the feedback sensor to enhance image stability and quality.

16. A method for performing optical coherence tomography imaging, the method comprising:
emitting a beam of light from a laser source;
continuously or discontinuously rotating the beam of light using a rotator-derotator mechanism to scan the beam across a sample;
capturing interference data of the light reflected from the sample during the rotation;
processing the captured data to reconstruct a real-time three-dimensional image of the sample.
